(19) United States
(12) Patent Application Publication
(10) Pub. No.: US 2019/0275201 A1
(43) Pub. Date: Sep. 12, 2019

(54) PHOTOPOLYMERIZABLE BONE FILLER MATERIAL

(71) Applicant: ECOLE POLYTECHNIQUE FEDERALE DE LAUSANNE (EPFL), Lausanne (CH)

(72) Inventors: Andreas SCHMOCKER, Lausanne (CH); Oriane POUPART, Richardmenil (FR); Dominique PIOLETTI, Buchillon (CH)

(21) Appl. No.: 16/349,834

(22) PCT Filed: Nov. 15, 2017
(86) PCT No.: PCT/IB2017/057124

§ 371 Date: May 14, 2019

(30) Foreign Application Priority Data
Nov. 16, 2016 (EP) 16199055.1

(51) Int. Cl. A61L 27/18; A61L 27/48; A61L 27/50

(52) U.S. Cl. CPC A61L 27/18; A61L 27/48; A61L 27/50; A61L 2400/06; C08L 33/12; A61L 2430/12; A61L 2430/38; C08L 2203/02; A61L 2430/02

(57) ABSTRACT

A photopolymerizable bone filler material comprising a fluid polymeric material, a photoinitiator and a plurality of solid, polymeric optical fibers is disclosed, wherein said optical fibers are used for both illumination and reinforcement of the entire photopolymerized structure. In one embodiment, a mixture of PMMA and MMA is used as the fluid polymeric material. In one embodiment, the photoinitiator belongs to the bisacylphosphine oxide (BAPO) family, providing a complete, quick and reliable polymerization of a PMMA/MMA liquid bone cement upon application of a light of suitable wavelength. Contrary to other photoinitiators and chemically-similar compounds used in an experimental setting, the BAPO photoinitiator permits to obtain a polymerized bone cement which is mechanically robust and suitable for in vivo applications. Methods for using the developed fluid bone filler are also herein disclosed.

### TECHNICAL FIELD

The invention lies in the field of filler, reinforcement and tissue replacement materials for biomedical applications and a device and a method to apply such materials.

### BACKGROUND ART

During medical surgery it is common to replace or reinforce tissue. To do so different materials such as cements, sealants, and fillers are used. A common example of such a filler material is bone cement to be placed into vertebrae after vertebral fractures. Vertebral fractures are a major health concern and the most common complication of osteoporosis worldwide. Vertebral compression fractures (VCFs) occur when a vertebral body is overloaded and collapses. VCFs are classified into three types: wedge (more than 50% of all VCFs), biconcave or end-plate (17%) and crush (13%). They may occur in the whole spine, but particularly in the lower part of the thoracic spine. A major cause of VCFs is trauma (car accident, sport injury . . . ) or metastatic tumors.

If possible, conservative therapy is the preferred treatment method of VCFs. Conservative treatments include short period of bed rest, pain control (analgesics), immobilization with orthoses and rehabilitation. Most patients heal after conservative treatment, however a surgical intervention may be necessary in case of back pain failing conservative treatment or severe fractures. Two percutaneous minimally invasive vertebral augmentation techniques, vertebroplasty and kyphoplasty, are currently the favoured methods.

Vertebroplasty consists of injecting a bone cement into the vertebral body under the guidance of a CT scanner and/or fluoroscopy in order to visualize the needle position, the cement position and cement distribution. The cement allows replacing damaged or missing bone and leads to a stabilization and reinforcement of the collapsed vertebral body. Kyphoplasty is a similar surgical method. The only difference is the introduction of an inflatable balloon which is then filled by the cement.

Most commercially available bone cements for vertebroplasty are based on polymethylmethacrylate (PMMA). PMMA cements are two-component systems consisting of a powder and a liquid which need to be mixed before surgery. Table 1 regroups the composition, function and proportion of the different components.

TABLE 1
Composition of commercial PMMA bone cements: two-component system

| | Composition | Functions | Components | Proportion by weight |
|---|---|---|---|---|
| Powder | Polymer | | PMMA | 83 to 99% |
| | Initiator | Catalyses polymerization | BPO | 0.75 to 2.6% |
| | Radio-opacifier | Makes the cement radio-opaque | BaSO4 or ZrO2 | 8 to 15% |
| | Antibiotics | Antimicrobial prophylaxis | | |
| Liquid | Monomer | | MMA | 98-99% |
| | Accelerator/Activator | Initiates cold curing of the polymer | DMPT | 0.8 to 2.5% |
| | Stabilizer/Inhibitor | Prevents early polymerization | Hydroquinone | 20 to 80 ppm |

After mixing powder and liquid, the initiator benzoyl peroxide (BPO) and the accelerator N,N-dimethyl-p-toluidine (DMPT) reacts together in order to produce free radicals which cross-link PMMA and methylmethacrylate (MMA) molecules. The benzoyl radicals attack the double bonds of the MMA monomer, leading to a free radical MMA monomer. This radical reacts with another MMA monomer or PMMA polymer and propagates until the formation of high molecular weight PMMA polymer.

The cement preparation and application can be divided in four steps during which the viscosity of the cement changes continuously:
the mixing phase where the powder and liquid are mixed together and homogenized (up to 2 minutes)
the waiting phase to obtain a dough-like and non-sticky cement (up to several minutes)
the working phase where the surgeon can inject the cement (2-10 minutes)
the hardening phase (2-15 minutes)

An example of the duration of the different phases of a commercial bone cement (cemSys3, Mathys European Orthopaedics) is presented in FIG. 1.

The mixing and polymerization speed of the cement determines the surgery timing and procedure arrangement. Between mixing and final hardening of the cement, the viscosity changes continuously in a non-linear manner. In most cases increasing slowly at the beginning, fast during an intermediate phase (often the working phase) and again slowly at the very end (during or after the hardening phase). The viscosity is influenced by the cement composition, the powder/liquid-ratio as well as the initiator and accelerator concentrations. It can be determined by a rheology test such as an intrusion test, which consists of compressing the cement in a perforated mold and measuring the cement extent of intrusion into the perforations. Other rheometers such as shear rheometers can be used as well as tests for viscosity and flow measurements.

For certain applications low- or high-viscosity cements are used. Low viscosity cements have a long waiting phase and high viscosity cements have a short waiting phase which is followed by a long working phase. Currently, the surgeon decides when to mix and then apply a cement depending on the indication of the patient's anatomy. The surgeon waits until the cement reaches the viscosity he wants to use.

Most commercially available cements do not have a constant viscosity, require the mixing of two compounds and can only be applied during a given period of time. Ideally, a cement has a constant, tuneable viscosity, does not required to be mixed and can be applied or eventually taken out after an unsuccessful application.

Young's modulus of the vertebral cancellous bone is several hundreds of megapascals. El Masri et al. [Computer methods in biomechanics and biomedical engineering, 15.1 (2012), pp. 23-8] demonstrated a mean value of 374±208 MPa (ranging from 87 to 791 MPa). Young's modulus of the vertebral cancellous bone depends on the age of the patient and on the location within the spine (Nicholson et al. [Medical Engineering & Physics 19.8 (1997), pp. 729-737] measured a mean Young's modulus of 165 MPa in the supero-inferior direction and 43 MPa in the lateral direction).

Although the percutaneous vertebral augmentation methods are useful interventions, these techniques have certain drawbacks. The polymerization of MMA is an exothermic reaction: there is a heat formation of 52 kJ per mole of MMA, which equals to 1.4 to 1.7 10⁸ J per m³ of cement. Ex vivo studies have showed temperature ranges between 45 to 115° C. (S. M. Belkoff and S. Molloy, Spine 28.14 (July 2003), pp. 1555-9). But the temperature elevation is lower in vivo. Anselmetti et al. (CardioVascular and Interventional Radiology 32.3 (2009), pp. 491-498) recorded peak temperatures of 44.8° C. for most of the tested cements. This heat production can cause local cell morbidity and necrosis of tissue surrounding the cement. The temperature elevation may be considered as an advantage only if the patient has metastatic tumors.

The MMA conversion in commercial cements does not reach 100%. Usually, 2 to 6% of residual monomer remain in an unreacted and still active state. Rudigier et al. showed that residual monomer polymerized mainly in the 24 hours following the surgery, decreasing the residual monomer content. However, unreacted MMA can still be released and lead to bone necrosis.

Another drawback is the volumetric shrinkage of the cement after solidification. The shrinkage of pure MMA is 21% but since bone cements are not totally composed of MMA, the cement shrinkage is approximately 6%. This polymerization characteristic can compromise the consolidation of the vertebral body and the bone/cement interface.

As previously mentioned, the surgeries need to be planned exactly according to the preparation of the cement. Once the two-components are mixed together, the polymerization cannot be stopped. The surgeon has to wait in order to get the appropriate viscosity and then inject the cement in a very short period. Therefore, the chemical polymerization is an issue since it cannot be controlled.

Photopolymerization uses the same polymerization mechanism than conventional polymerization except that the initiation is done with a photoinitiator and light illumination. Photoinitiators are molecules that absorb light at specific wavelengths. The interaction between the light and the initiator generates free radicals, ion radicals, cations or anions which then initiate the polymerization reaction. Photopolymerization, in comparison with thermally or chemically initiated polymerization, offers many advantages such as a spatial and temporal control, minimal heat production, rapid polymerization rates and high reaction rates at room temperature. Therefore, photopolymerization of bone cements could be a promising solution to deal with the drawbacks of current vertebroplasty procedures.

### SUMMARY OF INVENTION

In view of the drawbacks of the prior art in the field of bone fillers or cements, and the need for a surgeon to tightly regulate the polymerization rate in the frame of a bone surgery and vertebroplasty in particular, the inventors developed a new solution able to efficiently and advantageously overcome those issues using light-induced polymerization.

One aim of the present invention was to develop a material and a method for filling a bone void or repair a bone fracture in a surgeon-friendly manner.

Another aim of the present invention was to develop a mechanically-suitable, long-lasting stable bone cement by minimally altering the materials currently used in the clinic.

A further aim of the present invention was to efficiently and quickly photopolymerize a bone cement in a minimally-invasive procedure.

A further aim of the present invention was to develop a biocompatible bone cement with possibly a reduced cytotoxicity.

Still a further aim of the present invention was to possibly reinforce the obtained bone cement structure without altering the chemical composition thereof.

The developed bone filler material was designed in order to optimize it in terms of photopolymerization time, mechanical properties and biocompatibility. Said filler material is photopolymerizable viscous bone filler material comprising
a fluid polymeric material;
a photoinitiator; and
a plurality of solid, polymeric optical fibers, wherein said fibers comprise a core and a shell composed of two or more materials having two different indices of refraction.

In one implemented embodiment, the fluid polymeric material is based on a PMMA/MMA fluid mixture in which a phosphorus-based photopolymerization initiator was included. The material proved to be efficiently photopolymerized in terms of photopolymerization time and mechanical properties. The photoactivated cement implanted into osteoporotic bone models showed very similar performance compared to a commercial bone cement under mechanical loading. A medical device to inject and illuminate the bone filler material in situ is further disclosed herein.

In an embodiment the material is injected together with one or preferably several optical fibers, said optical fibers being placed parallel to the injection flow. The optical fibers incorporated into the material increase the polymerization rate of the entire volume. After polymerization they are left within the material or may as well be pulled out.

In one embodiment the core of the optical fiber and the fluid polymeric material of the bone filler consist of the very same material.

In a particular embodiment, PMMA optical fibers have been used for the illumination, which are polymerized into the bone filler and thus are eventually left within the polymerized filler at the end of the illumination/polymerization procedure so that these fibers eventually reinforce the entire polymerized structure.

To validate the entire system, the photo-cross-linked cement was implanted into an osteoporotic bone model using the developed device and evaluated under loading.

As for other photopolymerizable materials, the advantage provided by the presently developed bone filler composition relies on the fact that the viscosity of the material remains constant until the photopolymerization is started. This allows the surgeon to work with a handable material without being limited by the polymerization time upon PMMA/MMA mixing. Moreover, the choice of a particular family of photoinitiators permits to carry out the complete curing procedure in a short time (even shorter than with other photoinitiators) while guaranteeing a suitable ultimate strength of the bone cement.

It is therefore an object of the present invention to provide for an injection device comprising
a photopolymerizable viscous bone filler material comprising
i) a fluid polymeric material; and
ii) a photoinitiator; and
one or a plurality of solid, polymeric optical fibers, wherein said fibers comprise a core and a shell composed of two or more materials having two different indices of refraction.

In one embodiment, the bone filler material has a viscosity comprised between 10 Pa*s and 10⁶ Pa*s, preferably between 100 Pa*s and 10⁴ Pa*s.

In one embodiment, the light attenuation coefficient of the fibers is comprised between 10 dB/cm and 10⁻⁸ dB/cm.

In one embodiment, the optical fiber and the fluid polymeric material are substantially composed of the same material.

In one embodiment, the fluid polymeric material comprises a mixture of PMMA and MMA in a fluid state.

In one embodiment, the PMMA/MMA weight ratio is comprised between 0.5 and 4, preferably between 0.8 and 1.4, even more preferably of 1.

In one embodiment, the photoinitiator belongs to the bisacylphosphine oxide (BAPO) family.

the photoinitiator has the formula

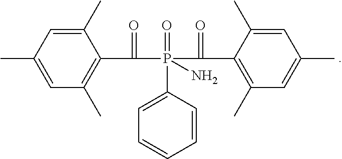

In one embodiment, the photoinitiator has a concentration comprised between 0.001 and 1 wt %, preferably 0.1 wt %.

In one embodiment, the bone filler material further comprises a radiopaque material.

In one embodiment, the optical fibers are PMMA optical fibers.

A further object of the present invention relates to the use of a bisacylphosphine oxide photoinitiator of the formula

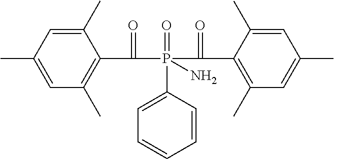

for the photopolymerization of a PMMA/MMA bone filler material.

Still a further object of the present invention relates to a method for treating a subject having a bone defect comprising the following steps:

a) providing the device as previously described;

b) injecting the bone filler material into or onto the bone defect; and

c) delivering into the injected photopolymerizable bone filler material an actinic light adapted to photopolymerize it through the optical fibers.

In one embodiment, the actinic light used to photopolymerize the bone filler material has a wavelength comprised between 300 and 550 nm, preferably between 400 and 450 nm.

In one embodiment, the light used to photopolymerize the bone filler material is delivered for a maximum of five minutes, preferably for a maximum of two minutes.

In one embodiment, the optical fibers are aligned parallel to the injection flow.

In one embodiment, the method further comprises a step of releasing the optical fibers or a portion thereof into the photopolymerized bone filler material.

In one embodiment, the bone defect is a bone fracture, a vertebral fracture or a dental defect.

Still a further object of the present invention relates to an implant obtainable by the above-described method, comprising a photopolymerized bone filler material and one or a plurality of solid, polymeric optical fibers.

### BRIEF DESCRIPTION OF DRAWINGS

In the Figures:

FIG. 1 represents the duration of the different handling phases of the cement cemSys3 from Mathys European Orthopaedics;

FIG. 2 illustrates the hardening procedure and impact of the viscosity of bone cements on the procedure

FIG. 3 shows the chemical structures of the photoinitiators used in the experimental phase: a) Irgacure 2959 b) Irgacure 819 c) BAPO-NH2 d) Camphorquinone e) Rose bengal f) Riboflavin;

FIGS. 4 and 5 depicts two embodiments of the medical device developed for the injection and photopolymerization of bone cements: Medical device Prototype 1 (FIG. 4): holes to insert optical fibers drilled in the syringe; Medical device prototype 2 (FIG. 5): holes to insert optical fibers drilled in the plunger;

FIG. 6 shows a schematic illustration (left) and a photography (right) of the cement injection and illumination with 250 μm PMMA optical fibers;

FIG. 7 depicts a schematic of the compression setup of Sawbones samples: the cavity is placed in horizontal position in order to simulate in vivo conditions of a bone fracture;

FIG. 8 shows the viscosity of Mathys bone cement over the different handling phases of the cement;

FIG. 9 shows the viscosity of the liquid cement for different PMMA/MMA-ratios;

FIG. 10 shows a graph concerning the viscosity stability over time for cements having a PMMA/MMA ratio of 1 and 0.8;

FIG. 11 shows a graph concerning the photorheology results for the different photoinitiators;

FIG. 12 shows 0.1% BAPO-NH₂-photopolymerized specimens before (left) and after (right) compression testing;

FIG. 13 shows the results of the compression test in function of photoinitiator used, its concentrations and illumination time;

FIG. 14 presents the extrusion pressure versus days after the cement preparation for PMMA/MMA ratio 1.5

FIG. 15 shows the viability results for non-polymerized cements (ratios PMMA/MMA of 1, 1.5 and 2) after 10 min, 30 min, 1 h and 4 h of exposure to the cells a) Measurement 1 h after the cement exposure b) Measurement 1 day after the cement exposure (1 day of incubation time of cells)

FIG. 16 illustrates the cell viability during photopolymerization of the cements (liquid state to solid state)

FIG. 17 show the cell survival/toxicity of polymerized cements measured at 7 days of exposure to the polymerized cements

FIG. 18 shows Giemsa staining microscopic images for evaluating the cytotoxicity of the used bone cements: a) Cells control b) Interface with BAPO-NH₂-photopolymerized cement c) Interface with Irgacure 819-photopolymerized cement d) Interface with camphorquinone-photopolymerized cement;

FIG. 19 shows specimens after compression according to the test conditions (without cement, with Mathys cement and with BAPO-NH₂-photoactivated cement);

FIG. 20 shows a graph of results of the compression testing on the different conditions specimens (empty cavity or filled with Mathys cement or BAPO-NH₂-photoactivated cement);

FIG. 21 shows a graph concerning the compressive stress at 10 and 25% strain for the different test conditions (without cement, with Mathys cement and with BAPO-NH₂-photoactivated cement).

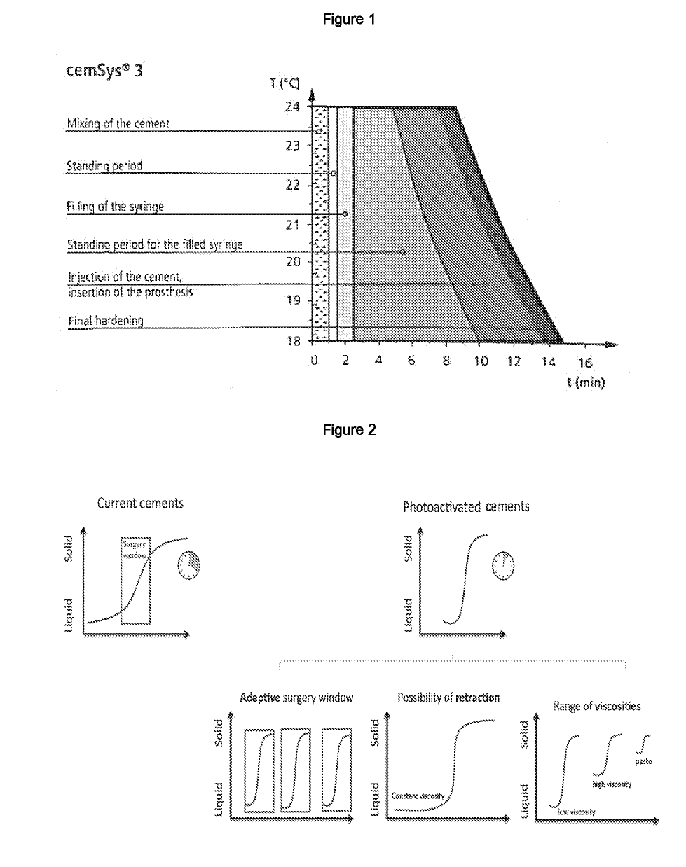

### DESCRIPTION OF EMBODIMENTS

The present disclosure may be more readily understood by reference to the following detailed description presented in connection with the accompanying drawing figures, which form a part of this disclosure. It is to be understood that this disclosure is not limited to the specific conditions or parameters described and/or shown herein, and that the terminology used herein is for the purpose of describing particular embodiments by way of example only and is not intended to be limiting of the claimed disclosure.

As used herein and in the appended claims, the singular forms “a”, “an” and “the” include plural referents unless the context clearly dictates otherwise. Thus, for example, reference to “an optical fiber” may include a plurality of such fibers and reference to “a radiopaque material” includes reference to one or more radiopaque materials, and so forth.

Also, the use of “or” means “and/or” unless stated otherwise. Similarly, “comprise”, “comprises”, “comprising”, “include”, “includes” and “including” are interchangeable and not intended to be limiting. It is to be further understood that where descriptions of various embodiments use the term “comprising”, those skilled in the art would understand that in some specific instances, an embodiment can be alternatively described using language “consisting essentially of” or “consisting of.”

The present invention is based, at least in part, on the intuition that, in the frame of the use of a photopolymerizable material suitable as a bone filler or cement, optical fibers can be used for both illumination and reinforcement of the entire photopolymerized structure. In particular, the invention features methods and materials in which advantageously optical fibers can be inserted upon surgical procedures for filling bone cavities or defects with the aim of photopolymerizing an injectable, fluid curable bone filler, and are later on released within the photopolymerized bone filler for reinforcing purposes. In one aspect of the invention, said procedure foresees the use of bundles of optical fibers that are parallel to the injection flow. In this context, an injection medical device has been designed by the inventors with the aim of facilitating the entire procedure, the device comprising a reservoir pre-filled with a biocompatible, injectable polymeric material suitable as a bone filler and one or preferably a plurality of optical fibers placed so to allow illumination of the bone filler upon injection out of the device. In preferred embodiments, the device is a pre-filled syringe.

The present invention is furthermore based, at least in part, on the surprising evidence that photoinitiators of the bisacylphosphine oxide family are able to induce the complete photopolymerization of PMMA/MMA bone cements volume of several cm³ in a rapid manner upon application of a light with a suitable wavelength. In this context, the used light is also referred to herewith as “actinic light”, i.e. a light to which a particular photosensitive material is sensitive; in other words, actinic light has the capacity to activate, polymerize or somehow alter the properties of a particular photosensitive material.

Up to the inventors' knowledge, this is the first report of the photopolymerization of a PMMA/MMA-based bone filler material (also referred to herein as “bone cement”) using visible light, as well as of the use of a bisacylphosphine oxide photoinitiator for promoting the photopolymerization of a PMMA/MMA-based bone cement. In particular, the photopolymerization of the PMMA/MMA-based bone filler material is such that the resulting bone cement is able to withstand compressive strength of several tens of MPa and a compressive stress at different strain percentage which is comparable to commercially available PMMA/MMA bone cements.

According to a preferred embodiment, the photoinitiator used is a modified version of the phosphine oxide, phenyl bis (2,4,6-trimethyl benzoyl) compound, also known as BAPO and currently commercially available with the tradename Irgacure 819. The used photoinitiator, renamed BAPO-NH₂, has the following chemical formula:

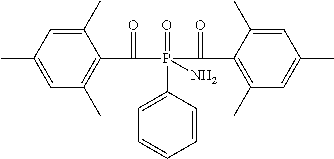

Surprisingly, the BAPO-NH₂ was able to allow the complete photopolymerization of PMMA/MMA bone cements in a quick and reliable manner, resulting in a final bone filler product which is suitable for use in a subject having a bone defect to be treated. Compared to commercially available cement it did not show any significant difference in terms of mechanical resistance (ultimate strength, FIG. 12). In this context, the PMMA/MMA fluid cement precursor comprising the BAPO-NH₂ photoinitiator photopolymerized in such a way that the resulting polymerized material showed superior mechanical features (e.g. higher ultimate strength, higher compressive strength or higher resistance to compressive stress) compared to the same bone filler comprising other photoinitiators.

Thus, the methods and compositions of the invention provide an efficient, safer and minimally invasive solution for the treatment of difficult clinical situations, such as the sealing of bone voids and defects as in case of a vertebral fracture, especially when specific viscosities are required, when the material needs to be extracted or retreated or when a full control of the solidification procedure is required. The composition described herein is useful in a variety of diseases, disorders, and defects where new bone formation and/or inhibition of bone resorption are an essential part of the therapy. In one embodiment it contains a bioactive molecule fostering bone growth or consists of a material fostering osteointegration of the cement.

Particularly, the bone filler composition according to the invention can be used for repairing long bone defects in the femur, tibia, fibula, and humerus and also for vertebral body defects, as in the case of a vertebral fracture. The composition could also be useful in periodontal diseases where the alveolar bone requires a support material for dental implants. Therefore, the photopolymerizable bone filler may be utilized for a variety of orthopedic, maxillo-facial and dental surgical procedures such as the repair of simple and compound fractures, non-unions requiring external or internal fixation, joint reconstructions and total joint replacements, repairs of the vertebral column including spinal fusion and internal fixation, tumor surgery, repair of spinal and vertebral injuries, intramedullary fixation of fractures, mentoplasty, temporomandibular joint replacement, alveolar ridge augmentation and reconstruction, inlay bone grafts and the like.

The bone cement according to the present invention is provided, in some embodiments, in a fluid, viscous formulation comprising a mixture of PMMA and MMA in an injectable, flowable fluid state having particular weight ratios. Said ratio can span from 0.5 to 4 depending on the sought viscosity for the bone cement, as will be detailed later on and in the Example section. In some preferred embodiments, a PMMA/MMA weight ratio is comprised between 0.8 and 3, preferably between 0.8 and 1.5, such as for instance a value of 1; these values have been chosen based on considerations regarding the viscosity stability over time and the spontaneous polymerization of the MMA monomers to form PMMA shown for higher PMMA/MMA weight ratio values as well as the comparison to commercially available bone cements which have a reaction between 2 and 3. By changing the PMMA polymer length or molecular weight as well as the initial grain size these ratios may vary. On strength of the disclosed invention is its ability to be easily adapted to almost any type of viscosity by changing polymer and monomer ration.

In some embodiments, the viscosity of the bone filler material of the invention is comprised between 10 Pa*s and 10⁶ Pa*s, preferably between 100 Pa*s and 10⁴ Pa*s, which is considered to be a suitable range for the injectability of the material in a minimally-invasive surgical context. Actually it has been shown that, for the selected PMMA/MMA weight ratios in the composition, the viscosity does not go beyond or below these values, which are perfectly suitable for a surgeon to work with. This allows to have a stable composition off the shelf.

The photoinitiator used can be present in the bone cement composition in an amount comprised between 0.001 and 1 wt %, such as between 0.01 and 1 wt %, with a preferred value being around 0.1 wt %, a value that experimentally proved to be ideal for the complete polymerization of the composition and a resulting cured bone cement of suitable mechanical properties (e.g. high compressive strength).

In one embodiment, the bone filler material of the invention comprises a radiopaque material. A “radiopaque material” is a material that contributes to at least the 70%, preferably to at least the 90%, of the radiopacity of the composition of the present invention. In most cases, said radiopaque materials are atoms or compounds comprising atoms having the highest atomic weight within the molecule, the compound or the material (in case several said radiopaque materials the second highest and so forth). In the frame of the present disclosure, the term “radiocontrast agent” can be interchangeably used to indicate radiopaque material. A general definition of a radiocontrast agent is a type of medical contrast medium used to improve the visibility of internal bodily structures in X-ray-based imaging techniques such as computed tomography (CT), radiography, and fluoroscopy. In another embodiment, contrast agents for other imaging techniques such as magnetic resonance imaging (MRI) are used.

The radiopaque material may be present in an amount of 5 to 20% w/w. In one embodiment, the radiopaque material comprises a metal, e.g. the radiopaque material may consist of or comprise metal or metalloid molecules, oxides and/or salts thereof. Examples of metal or metal-based radiopaque materials can be selected from a non-exhaustive list comprising barium sulphate, zirconium oxide, zinc oxide, calcium tungstate, gold, gadolinium, silver, iodine, platinum, tantalum as well as combinations of the foregoing or derivations thereof. Such derivation may include any type of molecular or atomic structure surrounding them or attached to them. The radiopaque material may also be provided in the form of particles having an average particle size in the micrometric or even nanometric scale.

One big advantage of the bone filler material of the present invention is its ability to be quickly and completely polymerized upon application of an actinic light of suitable wavelength and power. The polymerization process can be completed in up to ten minutes, ideally in up to five minutes, and even in maximum two minutes only, upon delivery within the composition of an electromagnetic radiation having a wavelength comprised between 300 and 550 nm, preferably between 400 and 450 nm and a total illumination power of 0.1 to 500 mW, ideally between 3 and 100 mW.

As will be evident, the invention also covers a method for treating a subject having a bone defect, such as e.g. a bone fracture, a vertebral fracture or a dental defect, comprising the following steps:

a) providing a photopolymerizable bone filler material comprising a photopolymerizable bone filler material as described, such as a filler material comprising a mixture of PMMA and MMA and a bisacylphosphine oxide (BAPO)-based photoinitiator of the formula

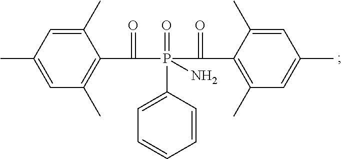

b) placing, preferably by injection, the photopolymerizable bone filler into or onto the bone defect; and

c) delivering into the photopolymerizable bone filler an actinic light adapted to photopolymerize it through optical fibers included into the bone filler material.

As already stated herein above, the actinic light used to photopolymerize the liquid bone cement has a wavelength comprised between 300 and 550 nm, preferably between 400 and 450 nm and has a total power of 0.1 to 500 mW, preferable 3 to 100 mW.

Advantageously, the light used for carrying out the photopolymerization process can be delivered into the fluid bone cement via one or a plurality of optical fibers or a bundle of optical fibers. In an implemented embodiment, the inventors adapted a syringe for injecting a liquid bone filler composition to have one or a plurality of optical fibers passing through the exit orifice of the said syringe (either a needle or a cannula connected thereto, or the exit bore of the syringe itself, opposite to the plunger) so that said fibers could easily access the bone defect and deliver the light to the injected liquid cement. Distally, the optical fibers are operably connected to a light source providing an actinic light of suitable wavelength.

In one embodiment the fiber or the fiber bundle is injected together with cement. In this embodiment the flow of the cement and the flow or the push direction of the fiber are parallel. Alternatively, an injecting device contains a second element or space where the cement flow and the push or fiber direction diverges and are not parallel anymore. In a preferred embodiment, there is a third element blocking the more rigid fiber thus exerting a compressive stress onto the fiber or the fiber bundle. This leads to a deformation of the fiber or fiber bundle, a lateral flexion or a coiling of the fiber or fiber bundle. In this preferred embodiment the 3D structure or arrangement of the fiber or fiber bundle is changed throughout the injection. The space or the volume between the fibers or the fiber bundles is changing as well throughout the injection and will be filled with the cement thus forming a heterogeneous 3D structure.

In one preferred embodiment, the method further comprises a step of releasing the optical fiber or a portion thereof into the photopolymerized bone cement. This possibility is tightly linked to the method itself, and can provide the additional advantage of obtaining a polymerized bone cement (i.e., a bone implant) comprising embedded therein optical fibers possibly functioning as reinforcing structures. In fact, upon polymerization of the fluid or otherwise liquid formulation, it can result difficult to remove the light transmitting elements, unless those are teared up from the injected composition as long as the polymerization procedure moves on. A solution could therefore be leaving them inside the cured bone cement and break the portions thereof which remained embedded into the photopolymerized bone cement. In some embodiments, the optical fibers can be PMMA optical fibers, so to keep the same chemical composition of the final bone cement and the biocompatibility. In this embodiment the PMMA cement may be covalently cross-linked to the fiber. However, as will be evident, optical fibers made of different material or mix of materials can be used in the frame of the disclosed method.

In one embodiment, the optical fibers further comprise means for favouring their breakage for the release into a photopolymerized bone filler implant. For instance, one or a plurality of grooves are placed along the body of the optical fiber so to facilitate the rupture thereof.

In a preferred embodiment, the curing agent is a photoinitiator. A “photoinitiator” is a molecule that creates reactive species (free radicals, cations or anions) when exposed to an electromagnetic radiation such as UV or visible light. Example of suitable visible or ultraviolet light-activated photoinitiator includes ITX 4-Isopropyl-9-thioxanthenone, Lucirin TPO 2,4,6-Trimethylbenzoyl-diphenyl-phosphineoxide, Irgacure 184 1-Hydroxy-cyclohexyl-phenyl-ketone, Irgacure 2959 1-[4-(2-Hydroxyethoxy)-phenyl]-2-hydroxy-2-methyl-1-propane-1-one, Irgacure 819 Phosphine oxide, phenyl bis (2,4,6-trimethyl benzoyl), LAP lithium phenyl-2,4,6-trimethylbenzoylphosphinate, Riboflavin 7,8-dimethyl-10-((2R,3R,4S)-2,3,4,5-tetrahydroxypentyl) benzo [g] pteridine-2,4 (3H,10H)-dione, Rose Bengal 4,5,6,7-tetrachloro-2′,4′,5′,7′-tetraiodofluorescein, PL-BDK Benzil dimethyl ketal, PL-CPK 1-hydroxy-cyclohexylphenyl-ketone, PL-HM PP 2-hydroxy-2-methyl-1-phenyl-1-propanone, Camphorquinone, 3-(4-Quantucure BPQ benzoylphenoxy)-2-hydroxy-N,N,N-trimethyl-1-propanaminium-chloride, APi-180 hydroxyalkylpropanone, bisacylphosphineoxide- or monoacylphosphineoxide-based initiators. In a preferred embodiment, a bis(acyl)phosphineoxide-derived (BAPO) photoinitiator such as bis(1,3,5-trimethylbenzoyl)phosphinic acid (BAPO-OH) is used. Other examples of suitable BAPO photoinitiators are given in the following references such as: K. Dietliker, A compilation of photoinitiators commercially available for UV today, SITA Technology Ltd, Edinbergh, London, 2002; J. V. Crivello, K. Dietliker, G. Bradley, Photoinitiators for free radical cationic & anionic photopolymerisation, John Wiley & Sons, Chichester, West Sussex, England, New York, 1998; S. Benedikt, J. Wang, M. Markovic, N. Moszner, K. Dietliker, A. Ovsianikov, H. Grützmacher, R. Liska, J. Polym. Sci., Part A: Polym. Chem. 2016, 54, 473-479; T. Majima, W. Schnabel, W. Weber, Makromol. Chem. 1991, 192, 2307-2315; S. Li, F. Wu, M. Li, E. Wang, Polymer 2005, 46, 11934-11939; M. A. Tasdelen, B. Karagoz, N. Bicak, Y. Yagci, Polymer Bulletin 2008, 59, 759-766; B. D. Fairbanks, M. P. Schwartz, C. N. Bowman, K. S. Anseth, Biomaterials 2009, 30, 6702-6707; A. Huber, A. Kuschel, T. Ott, G. Santiso-Quinones, D. Stein, J. Bräuer, R. Kissner, F. Krumeich, H. Schönberg, J. Levalois-Grützmacher, H. Grützmacher, Angew. Chem. 2012, 124, 4726-4730.G; Müller, M. Zalibera, G. Gescheidt, A. Rosenthal, G. Santiso-Quinones, K. Dietliker, H. Grützmacher, Macromol. Rapid Commun. 2015, 36, 553-557. The following patent applications are herewith full incorporated: WO 2005014605, WO 2006056541, WO 2011003772, WO 2014053455, WO 2014095724, EP 16189549.5

With the aim of favouring the polymerization of the composition, it is envisaged that methacrylate groups, diacrylate groups or the like are coupled to the polymeric cross-linkable material present in the carrier. Possible materials monomer or polymer or polymerizable materials are bis-GMA, bis-EMA, TEGDMA, Calcium phosphate, calcium sulphate, Hydroxyapatite and Ceramics particles (combeite). Suitable crosslinking agents can comprise for instance 1,4-Cyclohexanedimethanol divinyl ether, di(ethylene glycol) diacrylate, di(ethylene glycol) dimethacrylate, N,N′-(1,2-Dihydroxyethylene)bisacrylamide, divinylbenzene, p-Divinylbenzene, ethylene glycol diacrylate, ethylene glycol dimethacrylat, 1,6-Hexanediol diacrylate, 4,4′-M ethylenebis(cyclohexyl isocyanate), 1,4-Phenylenediacryloyl chloride, poly(ethylene glycol) diacrylate, poly(ethylene glycol) dimethacrylate, tetra(ethylene glycol) diacrylate or tetraethylene glycol dimethyl ether.

### EXAMPLES

Material Preparation

Similar as for commercially available bone cements, the photoactivated cement is composed of the monomer MMA, the polymer PMMA, a radiopacifier (Barium Sulfate) and an initiator system, in this case a photoinitiator. As previously mentioned, the viscosity is one of the most important parameters for the application of bone cements. This is why the first step was to determine the powder/liquid-ratio which mimics as close as possible the currently used bone cements' viscosity.

Photoinitiators are also an important component of the material since they initiate and activate the polymerization. Thus, in a second step, the study of different photoinitiators has been carried out in order to optimize the photopolymerization process.

PMMA/MMA-Ratio

PMMA powder (Mw=120,000 g/mol) and MMA were purchased from Sigma Aldrich. PMMA powder was added to MMA and the solution was homogenized by vortex for at least 4 hours. Different weight ratios of PMMA/MMA presented in Table 2 were prepared.

TABLE 2
PMMA/MMA-ratio samples

| Sample | 1 | 2 | 3 | 4 | 5 | 6 | 7 | 8 | 9 | 10 |
|---|---|---|---|---|---|---|---|---|---|---|
| PMMA/ | 2 | 1.33 | 1 | 0.8 | 0.67 | 0.5 | 0.4 | 0.33 | 0.25 | 0.125 |
| MMA-ratio | | | | | | | | | | |

Photoinitiators

Irgacure 2959 was purchased from BASF (Basel, Switzerland) and Irgacure 819 (or BAPO) was freely received from BASF. Camphorquinone, riboflavin and rose bengal were purchased from Sigma Aldrich. A BAPO-based photoinitiator, BAPO-NH₂, was synthetized. Chemical name and wavelengths in which photoinitiators absorb light can be found in table 3 and structures of the photoinitiators in FIG. 2.

TABLE 3
Photoinitiators chemical formula and ideal absorption wavelength

| | | Absorption |
|---|---|---|
| Photoinitiator name | Chemical formulation | wavelength (nm) |
| Irgacure 2959 | 1-[4-(2-Hydroxyethoxy)-phenyl]-2-hydroxy-2-methyl-1-propane-1-one | <370 |
| Irgacure 819 | Phosphine oxide, phenyl bis (2,4,6-trimethyl benzoyl) | 295, 370 |
| BAPO-NH₂ | | |
| Camphorquinone | | 450-490 |
| Rose bengal | 4.5,6,7-tetrachloro-2′,4′,5′,7′-tetraiodofluorescein | 555 |
| Riboflavin | 7,8-dimethyl-10-((2R,3R,4S)-2,3,4,5-tetrahydroxypentyl) | 365 |
| | benzo[g]pteridine-2,4(3H,10H)-dione | |

Photopolymerization of Samples

Photoinitiators and their concentration depended on the test. The photoinitiators used have been previously listed and the concentrations used are 0.01, 0.1 and 1 wt %.

Photoinitiator was added to MMA. PMMA (90 wt %) and Barium Sulfate (10 wt %) were dissolved in the solution with a powder/liquid-ratio of 1. The homogenization of the solution was done by vortex. Samples were wrapped in aluminum in order to prevent photopolymerization. To activate the photopolymerization process, material samples were illuminated with different lamps (Table 4). UV light lamps were firstly used in order to qualitatively evaluate the photopolymerization. A 405 nm laser was used for further tests because it was possible to couple the light through the medical device and because longer wavelengths are more bio-acceptable and induce less harm on cells.

TABLE 4
Different lamps used for cement illumination

| Lamp | Wavelength | Light power | |
|---|---|---|---|
| UVL-28 EL Series UV Lamp (UVP) | 365 nm | 3 | mW/cm² |
| Ligtningcure Spot LC8 mercury | UV and visible | 4500 | mW/cm² |
| lamp (Hamamatsu) | spectrum | | |
| Laser (Ebay) | 405 nm | 40 | mW/cm² |

Mechanical Testing

For benchmarking, mechanical testing was performed on the photoactivated bone cements and on the bone cement cemSys3 from Mathys European Orthopaedics. Rheology was used to test the material properties in the viscous state (“pre-op”-state), photorheology to access the transformation from viscous to solid (during implantation) and a compression test to evaluate the properties after solidification (“post-op”-state).

Rheology

Rheology was performed on different PMMA/MMA-ratios samples. An oscillatory time sweep experiment was performed on 1000 μm thickness samples with a Bohlin Instruments rheology machine. This rheology test consisted of studying the flow of the material by applying a constant 5% strain at 1 Hz frequency over time. Viscosity as well as elastic and viscous modulus were recorded during 240 seconds.

In order to determine the stability over time of the cements, the same test was performed at five days apart during 800 seconds to evaluate long-term changes of viscosity. Time sweep with the same parameter measurements was also performed for 3 samples of the bone cement cemSys3 during 15 minutes in order to record the viscosity during all the different phases (starting 2 minutes after mixing until the hardening phase).

Photorheology

Photorheology was performed with TA Instruments rheometer in order to quantitatively characterize the photopolymerization kinetics of the photopolymerized bone cement in function of each photoinitiator. The viscosity as well as the shear and elastic modulus were determined.

The illumination of a 1000 μm thickness sample was uniform across the area of the cement with an intensity of 3.5 mW/cm² at 405 nm. The upper plate was rotating at 1 Hz and applying a constant 5% strain on the sample.

Compression

Compression testing was performed in order to characterize mechanical properties of bone cements, in particular the compressive strength. Samples were cast in plastic molds with a diameter of 5.60 mm and thickness of 4.45 mm. The specimens were covered by microscope slides and illuminated by a 405 nm laser with an intensity of 45 mW/cm² during 2, 5 or 10 minutes.

Compression testing was performed with an Instron E3000 linear mechanical testing machine. A pre-load of 1 N was applied on the sample and then it was compressed at constant speed of 5 mm/minute. Load and displacement were recorded and ultimate compressive strength was calculated by the formula

$${σ}_{max} = \frac{{F}_{max}}{S}$$

where Fmax is the average ultimate load (or load applied to cause fracture) and S is the original cross-sectional area.

Medical Device Development

The medical device consists of a material and a surgery device for injection and an illumination system. In this section, the requirements are defined and the design of the surgery device is outlined. An essential part of the device are the optical fibers used for light delivery which are as well described in this section.

General Requirements

The photopolymerizable cement must be injected into the vertebral body in the viscous state and the illumination has to be performed in situ in order to activate the polymerization of the cement.

The following user needs were defined for the surgery device:

1. The cement can be used immediately (there is no two-component-mixing or waiting time); Moreover, the cement system is chemically stable over at least 18 months. The material within the device does not come in contact with light before the surgery and is not stored in a transparent package.

2. The surgeon can choose from different viscosities (material before illumination) according to the surgery which needs to be done;

3. The cement is delivered by an injection mechanism (e.g. a syringe); the light delivery system is embedded in the injection mechanism and can be switched on/off. A pressurization system is able to inject all cement viscosities.

4. The cement can be solidified by pressing a button; and

5. The solidification ideally takes no more than 5, ideally no more than 2 minutes.

Characterization of Optical Fibers

Various optical fibers were characterized in order to find the optimal fibers in terms of size and absorption properties. The optical fibers tested in this study include one PMMA optical fiber purchased from Swicofil AG and three fibers manufactured from Swiss Federal Laboratories for Materials Science and Technology (EMPA). Material and diameter of these fibers are specified in Table 5.

TABLE 5
Optical fibers

| Fiber | Material | Diameter (μm) |
|---|---|---|
| 1 | PMMA | 250 |
| EMPA 1 | cyclo-olefin (core) and fluoroplastic | 80 |
| | polymer (cladding) | |
| EMPA 2 | cyclo-olefin (core) and fluoroplastic | 80 |
| | polymer (cladding) | |
| EMPA 3 | silicon and polyurethane | 600 |

For the characterization, a 405 nm laser light was used. The collimated beam was coupled into the fibers through a convex lens (D=25.4 mm, f=25 mm). To ensure an optimal position of the focus on the proximal fiber end, three axis translational stage were used. The fibers were characterized by recording the input and output power for pieces of fiber ranging from 20 cm to 1 m (the increment was 20 cm).

Design of the Injection and Illumination Device

According to the requirements previously defined, two prototypes were made. Both prototypes were based on a conventional 3 mL (or lower or higher) syringe to accomplish the cement injection. Light delivery was achieved by the optical fibers, which were embedded into the syringe and were injected together with the cement. The insertion of the optical fibers into the syringe was investigated.

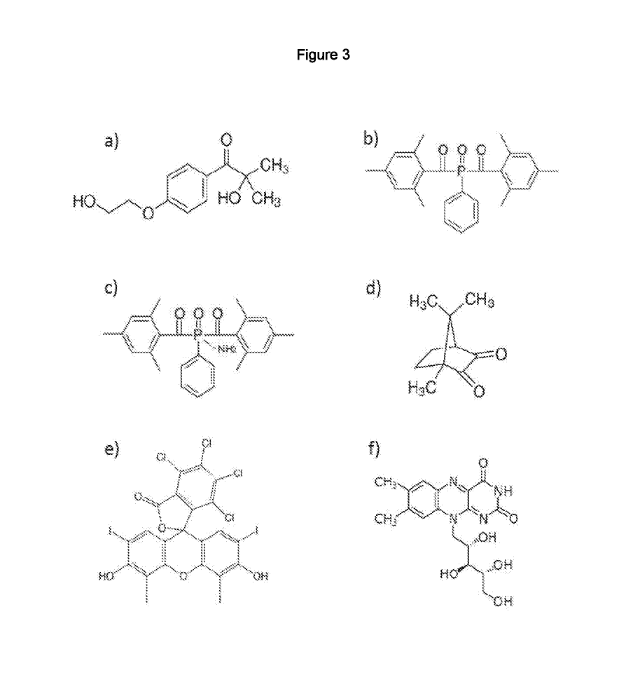

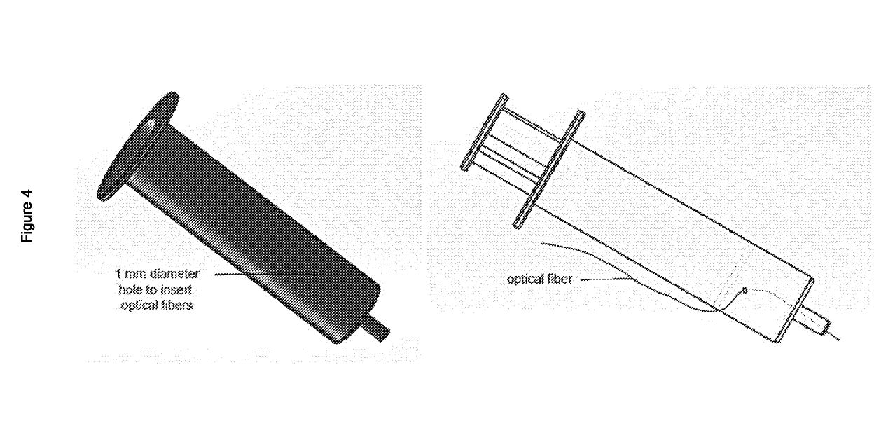

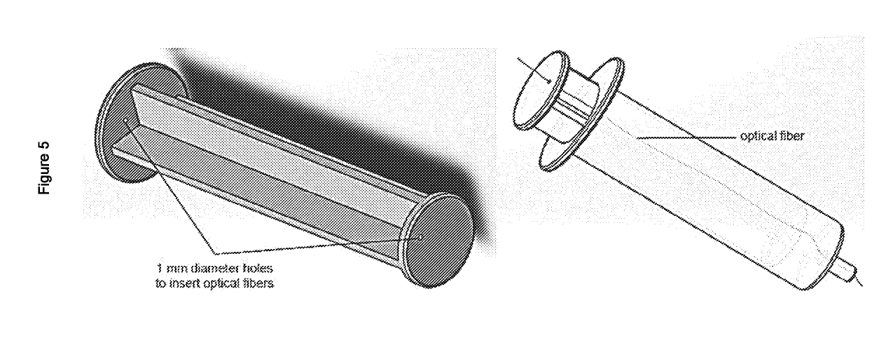

The prototype 1, shown in FIG. 3, consisted of drilling a 1 mm diameter hole in the syringe in order to insert the optical fibers. For the prototype 2, shown in FIG. 4, 1 mm diameter holes were drilled in the syringe piston. 250 μm PMMA optical fiber was inserted and glued using Loctite 3430 5 min epoxy adhesive. Light from a 405 nm laser was coupled into optical fiber embedded into the prototypes.

Stability of the Material

In order to study the stability of the material over time, an extrusion test was performed for the different ratios powder/liquid of the material. The extrusion test consisted of extruding the material at a constant rate of 0.1 mm/s through a small-sized hole and recording the force needed to inject the cement. This test has been performed at day 1, 7, 16 and 22 after mixing the cement. This test allows to determine the stability over time of the material and also the pressure needed for the material injection.

Biocompatibility Study: Viability Assay

Non Polymerized Cements

The biocompatibility study was performed on non-polymerized cements. A 96-well plate was filled with 3000 cells/mL and incubated during 3 days in order the cells to attach and to have 70-80% confluence. Cement sample was then put into each well during 15 min, 30 min, 1 h or 4 h and take it off, to determine up to which exposure time the material is not toxic for cells. Ratios PMMA/MMA of the samples were 1, 1.5 or 2. Viability was measured with Cell-Titer 96 Aqueous One Solution Cell Proliferation Assay at different times after the exposure to the cement material (1 h or 1 day later).

The biology of Cell-Titer 96 Aqueous One Solution Cell Proliferation Assay is described thereafter. The MTS tetrazolium compound is bioreduced by cells into a colored formazan product that is soluble in tissue culture medium. This conversion is presumably accomplished by NADPH or NADH produced by dehydrogenase enzymes in metabolically active cells.

Photoactivated cements viability results were compared with commercial cement (cemSys3 Mathys Orthopaedics) viability.

Liquid to Solid Cements Photopolymerization

A viability assay was also performed during the photopolymerization of liquid-state samples to solid-state. Liquid-state samples were placed in each well of the 96 well-plate during 5 or 30 min. Samples were then illuminated with a 405 nm laser at a 1.5 mW power during 5 min. The viability was measured, as previously using Cell-Titer 96 Aqueous One Solution Cell Proliferation Assay, 1 h, 1 day or 1 week after the illumination.

Polymerized Cements

The viability of polymerized cements was also studied using Cell-Titer 96 Aqueous One Solution Cell Proliferation Assay. For this test, cements samples were polymerized outside the cells with an 1.5 mW power illumination of 2 or 5 min. Then solid samples were placed on the 96 well-plate. Viability was measured 1 day or 1 week after exposure to solid cements. Cytotoxicity of the material using Gimsa staining was studied. The biocompatibility of the photopolymerized solid samples was investigated in bovine chondrocytes. Samples were surrounded by cells suspension and placed in the incubator at 37° C. and 5% CO₂ during 3 days. Giemsa surface staining protocol was then performed. Cement-cells interface was visualized using an inverted optical microscope (Zeiss Axiovert 100).

Table 2.1 summarizes the three viability assays and what parameters were evaluated.

TABLE 2.1
Parameters studied during viability assay of non-polymerized,
liquid to solid photopolymerization and polymerized cements

| Samples | Parameters | Parameters values |
|---|---|---|
| Non-polymerized | PMMA/MMA ratios | 1, 1.5 and 2 |
| cements | Exposure time to cells | 15 min, 30 min, 1 h and |
| | | 4 h |
| | Viability measurement time | 1 h and 1 day |
| Liquid to solid | Exposure time to cells | 5 min and 30 min |
| cements | Viability measurement time | 1 h, 1 day and 1 week |
| Polymerized | Photopolymerization time | 2 min and 5 min |
| cements | Viability measurement time | 1 day and 1 week |

Implantation of the Device

Sawbones Material Specimens Preparation

Sawbones material has been used in order to simulate the cortical bone of vertebral body. Sawbones material used in this study is polyurethane-based and has a density of 0.12 g/cm³. Specimens were cut into cubes with the dimensions of 20×20×20 mm. A cavity of 10 mm of diameter and 14 mm of depth was created. 3 types of conditions were applied to the samples:
injection of photoactivated cement using two 250 μm PMMA optical fibers. The total output light intensity was 35 mW/cm² and the fibers were placed at 4 mm and 10 mm deep into the cement. The illumination lasted 10 minutes. FIG. 5 schematizes the implantation of photoactivated cement into a Sawbones specimen and the light exposition by the optical fibers. The photoactivated cement was compared to the commercial Mathys cement.

Compression Testing

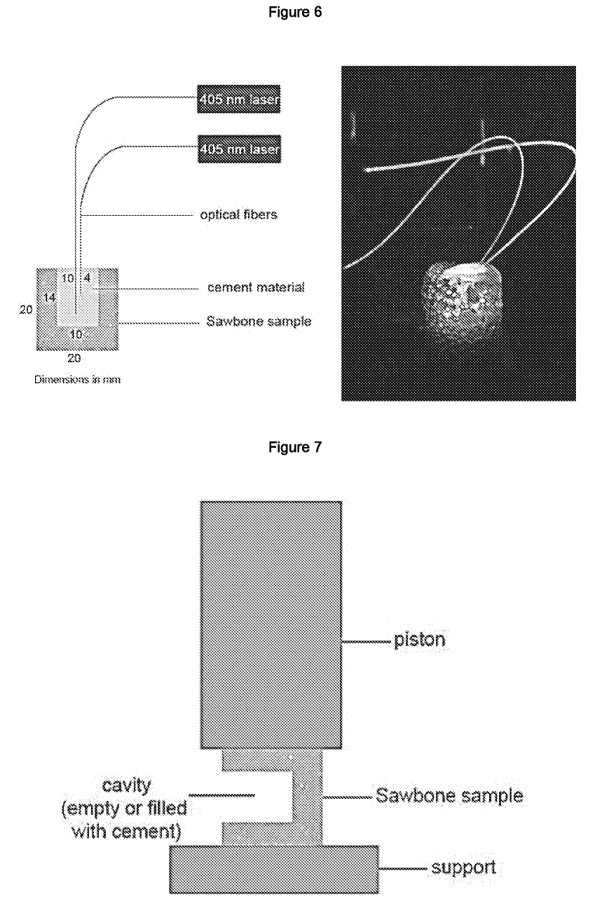

Compression testing was performed on Sawbones samples in order to simulate loads applied on the vertebrae and evaluate the influence of cement injection. To simulate in vivo conditions of a bone fracture, the cavity was placed in horizontal position, as shown in FIG. 6. In this position, the fracture (empty cavity) or the cement (filled cavity) was surrounded by Sawbones material. Similar to the compression protocol in cement materials previously described, a pre-load of 1N was applied on the Sawbones sample and then it was compressed at constant speed of 5 mm/minute. Ultimate compressive strength was recorded for each sample.

Statistics

Three specimens were tested for each condition. The statistical evaluation was done using Matlab. Results are expressed as mean±standard deviation. Paired t-tests were used for comparison. p<0.05 was considered as a significant result (denoted as *) and p>0.05 non-significant result (denoted ns). p<0.01 was denoted as ** and p<0.001 as ***.

Results

Viscosity

Viscosity of Commercial Cement

Rheology has been performed on Mathys cement in order to determine the viscosity at the different handling phases. This test showed a viscosity increase from 9.88 10²±154.7 Pa·s 2 minutes after mixing to 6.9499 10⁴±2.8068 10⁴ Pa·s at the final hardening phase of the cement, 13 minutes after mixing. Viscosity variation is showed in FIG. 7.

Influence of PMMA/MMA-Ratio on Photopolymerized Cement

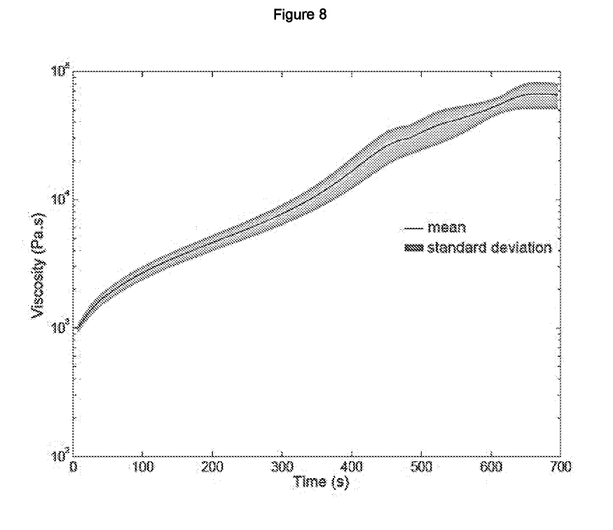

Rheology was used to measure the viscosity of the liquid cement (before injection) of different PMMA/MMA-ratios. The viscosity increases with increasing PMMA/MMA ratio and reaches a constant value around 10⁴ Pa·s for ratios between 0.8 and 1.4. Rheology results are presented in FIG. 8. To reach higher viscosities in the order of 10⁵ Pa·s, the ratio may be further increased.

Stability Over Time

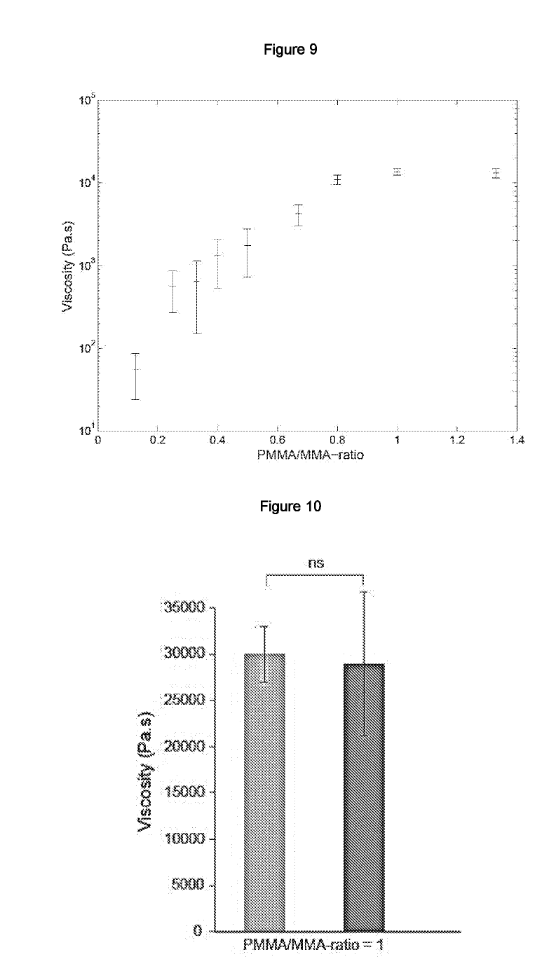

To study the stability of the liquid cement over time, we repeated the rheology test on specimen of ratio of 1 at five days apart. Viscosity results at 800 s are showed in FIG. 9. The viscosity is not significantly different for the ratio of 1 five days after mixing (p=0.812).

Evaluation of Photoinitiators

Curing performance of several photoinitiators was evaluated first using UV-light (at 365 nm or with mercury lamp as mentioned in Table 4). The material hardness was evaluated in order to determine if a polymerization occurred. Table 6 refers to the qualitative photopolymerization results only. All photoinitiators, except for rose bengal, allowed the polymerization after a few minutes. This qualitative pre-evaluation confirmed the possibility to photopolymerize cements and therefore to proceed further.

TABLE 6
Pre-evaluation of photoinitators curing performance
(* after longer illumination time)

| Photoinitiator | Photopolymerization |
|---|---|
| Irgacure 2959 | YES |
| Irgacure 819 | YES |
| BAPO-NH2 | YES |
| Camphorquinone | YES* |
| Riboflavin | YES* |
| Rose Bengal | NO |

The efficient photoinitiators have been characterized in photorheology. Photorheology testing has demonstrated a photopolymerization for Irgacure 819, BAPO-NH₂ and camphorquinone specimens. Samples with Irgacure 2959 and riboflavin did not polymerize after 30 minutes of illumination. Results are showed in FIG. 10.

Polymerization time and elastic modulus G′ vary according to the photoinitiator used. Table 7 summarizes the cross-linking characteristics of the different photoinitiators.

TABLE 7
Photopolymerization characteristics for several photoinitiators

| Photoinitiator | Photopolymerization time | |
|---|---|---|
| Irgacure 819 | 1 | min 30 |
| BAPO-NH2 | 1 | min |
| Camphorquinone | 15 | min |

| | |
|---|---|
| Irgacure 2959 | ∞ |
| Riboflavin | ∞ |

Mechanical Evaluation

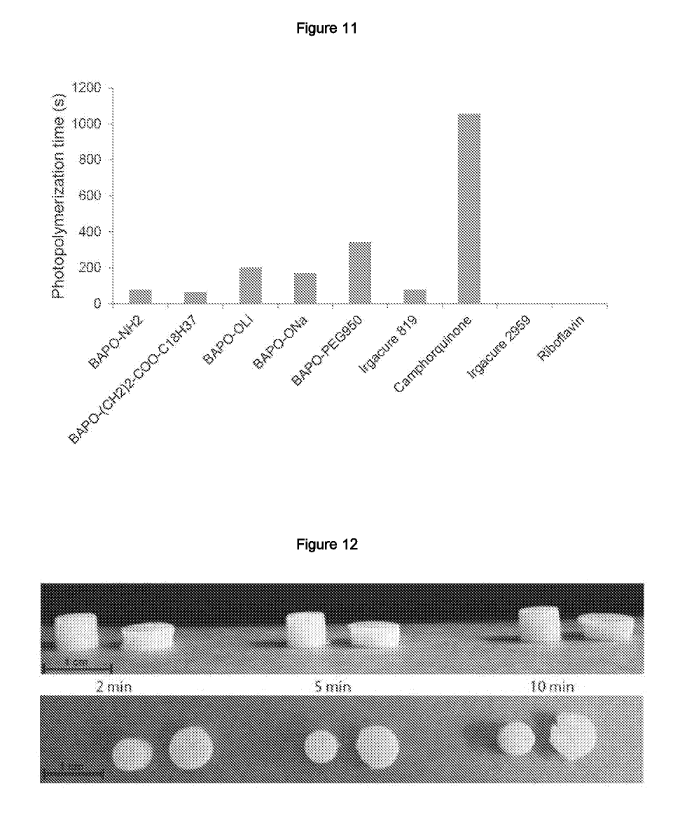

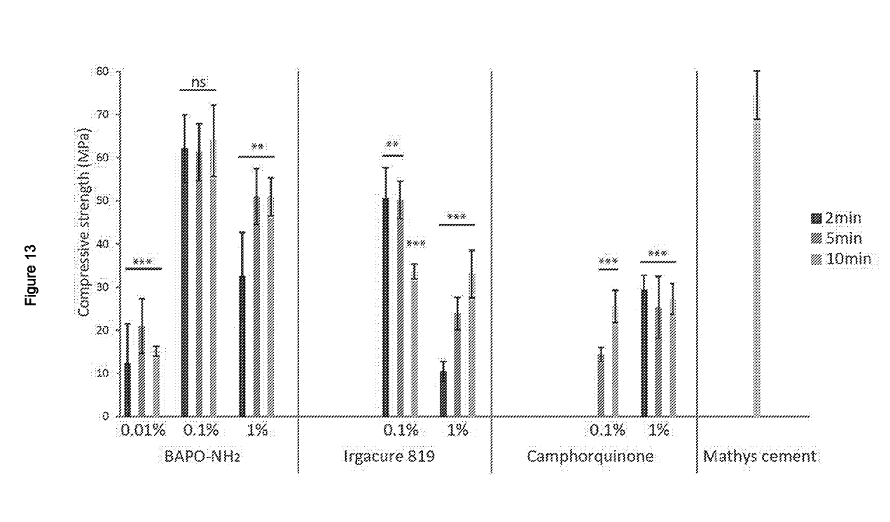

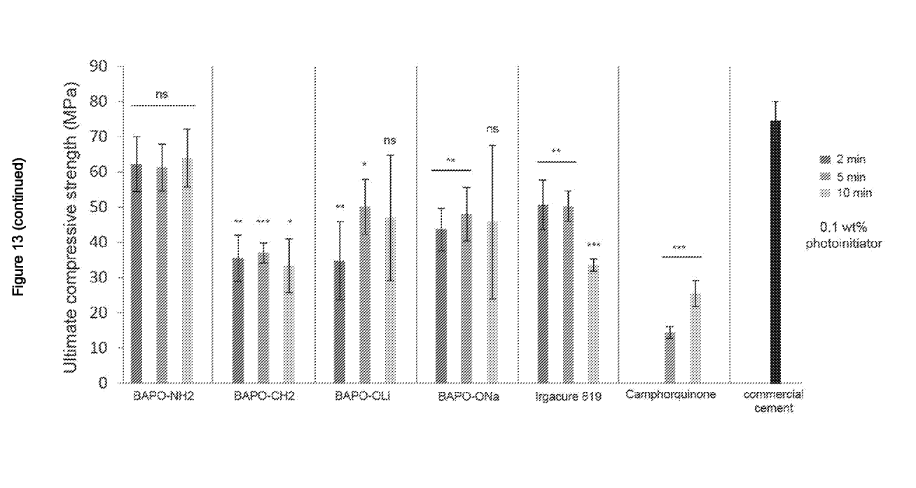

Based on the photorheology results, specimens with Irgacure 819, BAPO-NH₂ and camphorquinone were selected for compression testing. Tests were performed in function of photoinitiator concentrations and illumination time. FIG. 11 shows a representative set of specimens before and after compression. A shortening and lateral spread of the specimen can be observed after compression. FIG. 12 shows the mean and standard deviation of the ultimate compressive strength in function of illumination time and photoinitiator concentration for different photoinitiators. Paired t-tests were performed between each condition and Mathys cement (taken as a reference).

The comparison of photoinitiators in compression show that BAPO-NH₂ specimens present higher ultimate strength than Irgacure 819 and camphorquinone. In terms of photoinitiator concentration, it was observed that a concentration of 0.1% provides higher mechanical properties, except for camphorquinone where 1% is the optimum concentration. For Irgacure 819 and camphorquinone, specimens did not polymerize at a 0.01% concentration.

Illumination time influences the mechanical properties as well. In general, a longer illumination of the specimens led to higher compressive strength (e.g. the 0.1% camphorquinone specimens which did not polymerize after 2 minutes of illumination but polymerize after 5 minutes). For 0.1% BAPO-NH₂ samples, compressive strength is not significantly different for the different times of illumination. It can be concluded that the photopolymerization was completed after 2 minutes of illumination. This result is in accordance with photorheology results which present a faster photopolymerization for BAPO-NH₂.

The Mathys cement presents the highest rupture strength in comparison with all conditions. Only 0.1% BAPO-NH₂ mechanical properties are not significantly different (p>0.05) from the Mathys cement. Therefore, we it can be concluded that 0.1% BAPO-NH₂ is the most efficient photoinitiator to polymerize PMMA/MMA bone cement. For a volume of 1.1 cm3, 2 minutes of illumination is enough to complete the photopolymerization.

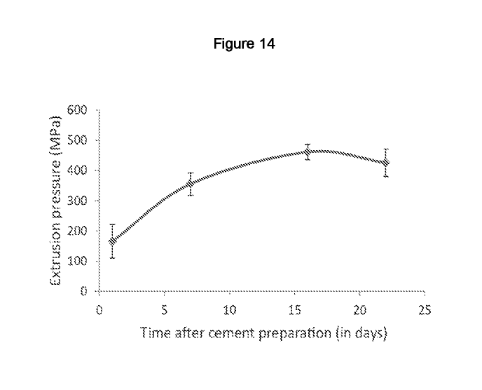

Unpolymerized precursor solutions, especially with photoinitiators inside can be unstable. The stability of the non-polymerized material in FIG. 14 was evaluated with an extrusion test. The PMMA/MMA ratio was 1.5. The extrusion pressure increases up to 15 days after the cement preparation, but then reaches a plateau at approximately 450 MPa.

Viability

Non Polymerized Cements

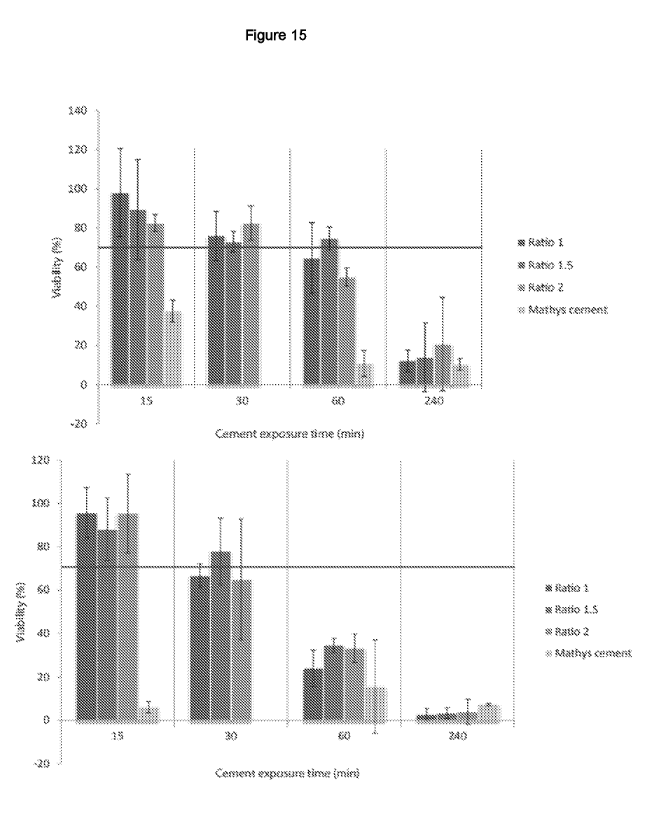

Viability results of non-polymerized cements, in FIG. 15, show three parameters influence: ratios PMMA/MMA, material exposure time to the cells and measurement time after exposure.

A viability greater than 70% is typically required in order to conclude that a material is biocompatible.

Results showed no significant influence on the ratio PMMA/MMA.

The viability of the material decreases when the exposure time to the cells increases. However, the photoactivated cements are viable up to 30 min exposure.

Similar results were found when viability was measured 1 h or 1 day after the exposure.

An interesting result is also the non-viability of the commercial cement independently to the exposure time or measurement time.

Therefore, the viability of photopolymerizable cements is higher than for commercial cements.

Liquid to Solid Cements Photopolymerization

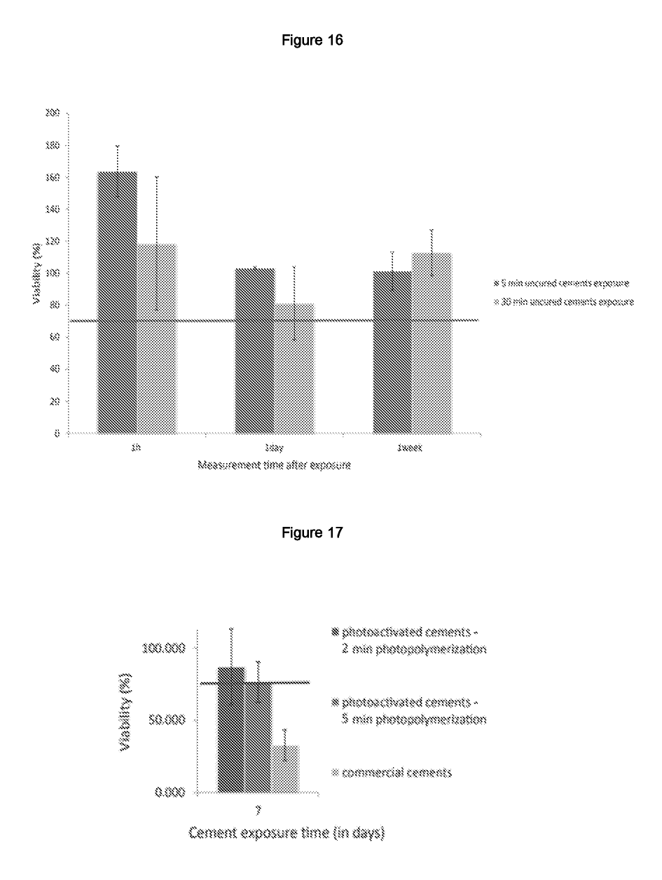

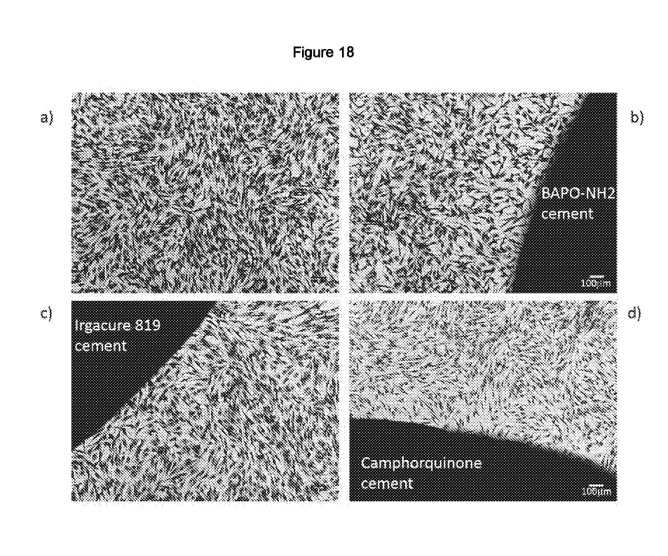

Bovine chondrocytes were exposed to uncured material during 5 or 30 min (FIG. 16). Then the material (on top of the cells) was illuminated during 5 min for cross-linking. Results showed that the material is viable up to 1 week independently to the exposure time. This indicates that the photopolymerization process does not have a negative impact on the cells during photopolymerization and illumination.

Polymerized Cements

Viability assay on bovine chondrocytes showed that polymerized cements are significantly less toxic up to 1 week exposure to cells when compared to commercial cements (FIG. 17).

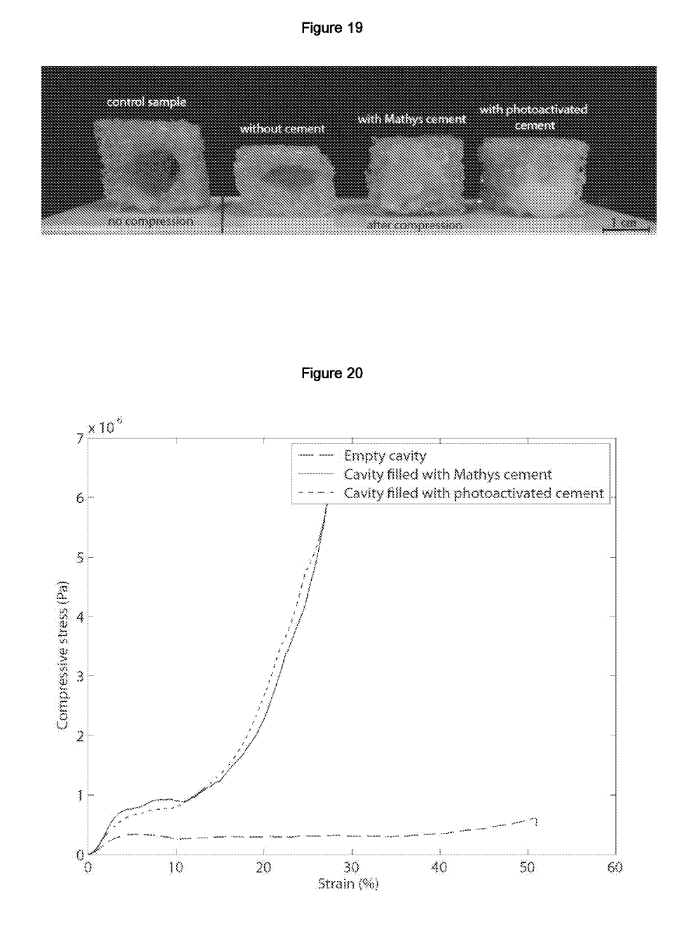

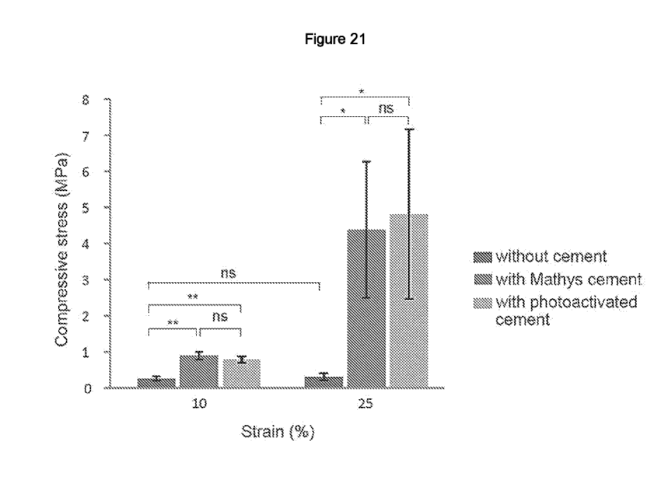

Giemsa staining is used to topographically colour the cells. Thus, the distribution of the cells surrounding the samples provides preliminary results about the biocompatibility of the photoactivated cements. Microscopic images of the cement-cells interface are presented in FIG. 19. No significant difference can be observed between the solution with only bovine chondrocytes and the interface cement-cells. The phopolymerized cements do not seem to be toxic for the surrounded cells independent of the used photoinitiator system.

Implantation of the Device

The implantation of the photoactivated cement into Sawbones material and the illumination by PMMA optical fibers were performed in order to validate the system. The compressive stress at 10% and 25% strain were compared between each test condition. FIG. 14 shows a set of compressed specimens without cement, with Mathys cement and with the photoactivated BAPO-NH₂ cement.

It can be observed that the thickness of both injected cement samples was higher than the sample without cement. The height of Mathys cement and photoactivated cement sample after compression was similar and not significantly different. Compression testing results are showed in FIG. 15. The photoactivated cement and Mathys cement have the same mechanical behavior in compression.

The compressive stress increases up to reaching a plateau of approximately 0.75 MPa when the Sawbones material start to be damaged and then the stress increases because of the strength of the cement. For the empty cavity, the compressive stress increases up to a plateau of 0.5 MPa. The comparison of the compressive stress at 10% and 25% strain, in FIG. 16, showed a significant rise of the compressive stress from 10 to 25% strain for both cements (p=0.041 for the photoactivated cement and p=0.033 for Mathys cement), while the results are not significantly different for the empty cavity (p=0.4659). The compressive stress of the photoactivated cement and Mathys cement are not significantly different (p=0.8149 at 25% strain). Thus, the photoactivated cement provides a good consolidation of osteoporotic bone models.

1. An injection device comprising
a photopolymerizable viscous bone filler material comprising
i) a fluid polymeric material; and
ii) a photoinitiator;
and
one or a plurality of solid, polymeric optical fibers, wherein said fibers comprise a core and a shell composed of two or more materials having two different indices of refraction.

2. The device according to claim 1 wherein the bone filler has a viscosity comprised between 10 Pa*s and 10⁶ Pa*s, preferably between 100 Pa*s and 10⁴ Pa*s.

3. The device according to claim 1, wherein the light attenuation coefficient of the fibers is comprised between 10 dB/cm and 10⁻⁸ dB/cm.

4. The device according to claim 1 wherein the optical fiber and the fluid polymeric material are substantially composed of the same material.

5. The device according to claim 1, wherein the fluid polymeric material comprises a mixture of PMMA and MMA in a fluid state.

6. The device according to claim 5, wherein the PMMA/MMA weight ratio is comprised between 0.5 and 4, preferably between 0.8 and 1.4, even more preferably of 1.

7. The device according to claim 1, wherein the photoinitiator belongs to the bisacylphosphine oxide (BAPO) family.

8. The device of claim 7, wherein the photoinitiator has the formula

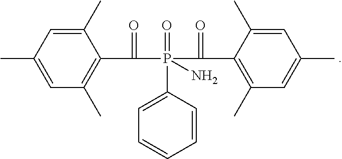

9. The device according to claim 1, wherein the photoinitiator has a concentration comprised between 0.001 and 1 wt %, preferably 0.1 wt %.

10. The device according to claim 1, wherein the bone filler material further comprises a radiopaque material.

11. The device according to claim 1, wherein the optical fibers are PMMA optical fibers.

12. A method for treating a subject having a bone defect comprising the following steps:
a) providing the device of claim 1;
b) injecting the bone filler material into or onto the bone defect; and
c) delivering into the injected photopolymerizable bone filler material an actinic light adapted to photopolymerize it through the optical fibers.

13. The method of claim 12, wherein the actinic light used to photopolymerize the bone filler material has a wavelength comprised between 300 and 550 nm, preferably between 400 and 450 nm.

14. The method of claim 12, wherein the light used to photopolymerize the bone filler material is delivered for a maximum of five minutes, preferably for a maximum of two minutes.

15. The method of claim 12, wherein the optical fibers are aligned parallel to the injection flow.

16. The method of claim 12 further comprising a step of releasing the optical fibers or a portion thereof into the photopolymerized bone filler material.

17. The method of claim 12, wherein the bone defect is a bone fracture, a vertebral fracture or a dental defect.

18. An implant obtainable by the method of claim 16, comprising a photopolymerized bone filler material and one or a plurality of solid, polymeric optical fibers.

19. A photopolymerizable bone filler material comprising a fluid polymeric material, a photoinitiator and a plurality of solid, polymeric optical fibers.

20. Process for manufacturing a bone filler material as defined in claim 19 wherein said optical fibers are distributed within the material in a way as to be used for light delivery and as reinforcement structure.
